(19) United States
(12) Patent Application Publication
(10) Pub. No.: US 2025/0127737 A1
(43) Pub. Date: Apr. 24, 2025

(54) NOVEL NAPROXEN SODIUM PREPARATIONS FOR PARENTERAL ADMINISTRATION

(71) Applicant: LEIUTIS PHARMACEUTICALS LLP, Hyderabad (IN)

(72) Inventors: Chandrashekhar Kocherlakota, Secunderabad (IN); Nagaraju Banda, Hyderabad (IN); Arjun Narala, Warangal (IN); Srinath Akula, Hyderabad (IN)

(21) Appl. No.: 18/832,654

(22) PCT Filed: Jan. 24, 2023
(86) PCT No.: PCT/IB2023/050579

§ 371 Date: Jul. 24, 2024

(30) Foreign Application Priority Data
Jan. 25, 2022 (IN) 202241004161

(51) Int. Cl. A61K 31/192; A61K 9/00; A61K 45/06; A61K 47/18; A61K 47/40

(52) U.S. Cl. CPC A61K 31/192; A61K 9/0019; A61K 45/06; A61K 47/183; A61K 47/40

(57) ABSTRACT

The present invention relates to a stable, parenteral formulations of naproxen sodium. The formulation further relates to liquid formulations comprising naproxen sodium, cyclodextrin and optionally other pharmaceutically acceptable excipients selected from pH modifying agents, chelating agents and tonicity agents. Also relates to the process of preparing the same. These formulations are stable when stored at recommended storage conditions and can be manufactured using simple manufacturing procedure. These compositions can be used to treat pain, particularly when quicker onset of action is desired. Further the present invention also relates to the parenteral formulations of naproxen in combination with additional active ingredients.

### CROSS-REFERENCE TO RELATED APPLICATION

This application is a Section 371 National Stage Application of International Application No. PCT/IB2022/050579, filed 24 Jan. 2023, and published as WO 2023/144692 on 3 Aug. 2023, in English, which claims priority to IN patent application Serial No. 202241004161, filed 25 Jan. 2022, the contents of which are hereby incorporated by reference in their entirety.

### FIELD OF THE INVENTION

The present invention relates to a stable, parenteral formulations of naproxen sodium. The invention also describes simple process for preparing such formulations.

### BACKGROUND OF THE INVENTION

Nonsteroidal anti-inflammatory drugs (NSAIDs) are the most commonly used therapeutically effective group of drugs that provide analgesic, antipyretic, anti-inflammatory effects. The main mechanism of action of NSAIDs is the inhibition of enzyme cyclooxygenase (COX). There are two cyclooxygenase isoenzymes, COX-1 and COX-2. COX-1 produced in the body plays a role in maintaining gastrointestinal mucosa lining, kidney function, and platelet aggregation, whereas COX-2 is expressed during an inflammatory response. Most of the NSAIDs are nonselective and inhibit both COX-1 and COX-2 resulting in gastric problems. So formulating a product using COX inhibitor that provides inflammatory relief without compromising the gastric mucosa is very important.

Naproxen is one of the non-selective Cyclooxygenase (COX) inhibitor with analgesic and antipyretic properties. It is a derivative of naphthyl propionic acid and the sodium salt form has the following formula:

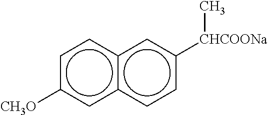

Commercially Naproxen sodium is available as tablets, capsules and suspensions. It is also used in combination with drugs such as sumatriptan, diphenhydramine hydrochloride, pseudoephedrine hydrochloride and proton pump inhibitors. It is approved for the treatment of rheumatoid arthritis, osteoarthrosis (degenerative arthritis), ankylosing spondylitis, acute musculoskeletal disorders, dysmenorrhoea, acute gout, management of pain like back ache, head ache, tooth ache, polyarticular juvenile idiopathic arthritis, tendonitis and bursitis.

The time taken for onset of action of naproxen is about an hour and peak plasma levels are obtained only after 2-4 hours. This may be a disadvantage when faster pain relief is desired. Hence there seems to be an unmet need to provide naproxen sodium in a parenteral dosage form with faster pain relief and reduced side effects.

But parenteral formulations of naproxen sodium are difficult to formulate, possibly due to issues with its physical and long term stability.

U.S. Application No. US2004105778 (A1) and U.S. Pat. No. 6,153,225 to Lee Robert et al., discloses lyophilized nanoparticulate formulations of naproxen, followed by terminal sterilization by gamma irradiation. The processes disclosed in this application require specialised equipment and long processing times. Here, the drug is present in a nanoparticulate form but not in a solution form. It is often difficult to formulate a cost effective product using these technologies.

CN Patent No. 100364524 (C) discloses a sterile, freeze dried parenteral formulation of naproxen. The document states that naproxen solutions for injection are unstable to conditions such as light, heat, acid, alkali and oxidations. Changes can also be seen in solution appearance, colour, content reduction and increase in impurities. The document proposes to make lyophilized formulations of naproxen to overcome the stability issues. The bulk solutions before lyophilisation were found to have a slight brown colour and a hemolysis factor greater than 10.

Loyd Allen discloses a composition of Naproxen sodium injection in journal US Pharm. 2009; 34(5):48-49. The composition contains propylene glycol, benzyl alcohol and sodium hydroxide/hydrochloric acid. The disclosure instructs that the injection is to be used within 24 hours if stored at room temperature and within 3 days if stored under refrigerated conditions. The maximum time period allowed is 14 days if the sterility is tested. This suggests that it is difficult to formulate a stable parenteral formulation of naproxen with long shelf life.

U.S. Pat. No. 5,128,373 teaches that it is possible to stabilize naproxen parenteral formulations only by adding compounds containing sulphydryl, sulfide or disulfide groups, together with polyhydroxylic alcohols, to the aqueous solutions of the sodium salt of the naproxen. The patent also suggests packaging the vials or the medicine-bottles containing these solutions in suitable containers made by boxes of polystyrene or of an equivalent material, like for instance polyvinylchloride, covered with films that prevent the light radiations from interacting with the substances dissolved. The compositions have high concentration of polyhydric alcohols and other excipients. For e.g., for delivering 100 mg naproxen dose, the quantity of excipients used in the formulation are 145.45 mg of polyhydric alcohols, 18.18 mg of N-Acetyl Cysteine and 7.27 mg of Lidocaine. Polyhydric alcohols when used in high concentrations in parenteral formulations over a short period may cause hyper osmolality, refractory hypotension, arrhythmias, hemolysis, renal dysfunction, seizure and coma. Paediatric patients also might develop CNS depression and seizures.

The formulations disclosed in the said patent were reproduced and tested by the inventors of the instant application. The formulation was found to be hazy and hypertonic which makes it unsuitable for parenteral application.

PCT Publication No. WO9532737 to Penkler et al., discloses oral compositions comprising inclusion complex of a beta-cyclodextrin with a sparingly water-soluble NSAID.

European Patent Publication No. EP1004318 to Yoshiaki et al., discloses injection compositions containing slightly water-soluble drug, a cyclodextrin and a water-soluble organic solvent.

The above prior arts fail to specifically disclose a solvent free stable parenteral formulations of naproxen in combination with cyclodextrin.

Thus the objective of the present invention is to formulate a stable solution of naproxen sodium for parenteral administration overcoming the disadvantages associated with the prior art.

### SUMMARY OF THE INVENTION

One aspect of the present invention is to provide a liquid parenteral formulation of naproxen sodium comprising cyclodextrin and an aqueous solvent.

Another aspect of the invention is to provide a stable liquid parenteral formulation of naproxen sodium comprising SBECD and an aqueous solvent.

Another aspect of the invention is to provide a stable liquid parenteral formulation of naproxen sodium comprising SBECD/cyclodextrin, an aqueous solvent, wherein the ratio of naproxen:cyclodextrin ranges from 0.5:1 to 11:1

Yet another aspect of the invention is to provide a stable liquid parenteral formulation of naproxen sodium of naproxen sodium comprising SBECD, one or more pH modifying agents and an aqueous solvent, wherein the formulation has drug content of more than 95% throughout shelf life.

Yet another aspect of the present invention is to provide a stable, parenteral formulation of naproxen sodium with total impurities less than 0.5% when placed at recommended storage conditions.

### DETAILED DESCRIPTION OF THE INVENTION

The term “naproxen” described in the present invention refers to pharmaceutically acceptable salts, solvates, hydrates, acids, anhydrous and free base forms thereof, preferably naproxen sodium.

The term “parenteral administration” described in the present invention refers to subcutaneous injection, intramuscular injection, intra-articular injection and intravenous injection.

The term “stable” means a formulation which has >95% of the drug remaining throughout its shelf life when stored at controlled room temperature in its primary pack made of glass or polyethylene or polypropylene or cyclo-olefin block polymers.

The inventors were successful in formulating a naproxen injection that is stable, non hemolytic and overcomes the disadvantages of prior arts. When compared to the formulations disclosed in the art, the present formulations have many advantages like:
(i) Reduced excipients and low excipient overload
(ii) Better hemolytic factor
(iii) Simple and cost effective manufacturing process
(iv) Better physico-chemical stability

A comparison of the instant formulation with the prior art can be better understood vide the experiments carried out by the inventors. The prior art composition disclosed in U.S. Pat. No. 5,128,373 is reproduced and checked for pH and osmolality.

TABLE 1
Composition from U.S. Pat. No. 5,128,373

| | | Quantity (mg/mL) |
|---|---|---|
| S. No | Ingredients | B#041K |
| 1 | N-Acetyl Cysteine | 12.5 |
| 2 | Propylene glycol | 100 |
| 3 | Naproxen Sodium | 68.75 |
| 4 | Lidocaine | 5 |
| 5 | Sterile Water for Injection | Qs to 1 mL |

Test Parameters

| | |
|---|---|
| (1) pH | 7.25 |
| (2) Osmolality (mOsm/Kg) | 1918 |

The formulation was found to be hazy and the osmolality was extremely high. Osmolality is a measure of number of particles of solute per unit of solution and is used to check the irritation potential of a parenteral formulation. The high osmolality of this formulation indicates that it is not suitable for parenteral administration as it may cause infection at the IV site, thrombophlebitis, extravasation, and hypervolemia.

Similarly, the formulations disclosed in CN100364524 were reproduced. The patent discloses lyophilised formulations which need a complex manufacturing equipment. The bulk solutions were reproduced and tested for hemolysis, colour change, pH and Osmolality.

TABLE 2
Compositions of CN100364524

| | | Quantity (mg/mL) | | |
|---|---|---|---|---|
| S. No | Ingredients | B#041E1 | B#041E2 | B#041E3 |
| 1 | Naproxen Sodium | 132.5 | 100 | 137.5 |
| 2 | Mannitol | 50 | 50 | 50 |
| 3 | Glucose | — | — | — |
| 4 | Sodium Hydroxide | Qs to pH | Qs to pH | Qs to pH |
| | | 8.8-9.2 | 8.8-9.2 | 8.8-9.2 |
| 5 | Sodium Citrate | — | — | — |
| 6 | Water for Injection | Qs to 1 mL | Qs to 1 mL | Qs to 1 mL |

| | | | |
|---|---|---|---|
| Test Parameters | | | |
| (1) % Hemolysis | 12.9 | 54.9 | 42.5 |
| (2) Colour of the Solution | 0.038 | 0.032 | 0.045 |
| (3) pH | 8.36 | 8.13 | 8.55 |
| (4) Osmolality (mOsm/Kg) | 687 | 693 | 675 |

Hemolytic factor relates to ability of compound to induce rupture of red blood cells (hemolysis) in the body. The hemolytic potential of the above formulations was many folds higher when compared with the present invention formulation (discussed subsequently). Generally, formulations with a hemolysis value of <10% are considered nonhemolytic and those with a value>25% have a definite hemolysis risk. As can be seen from the above data, none of the formulations have a hemolytic factor of less than 10 and two of those have values greater than 25%.

TABLE 3
The comparative results of the invention formulation
prepared as per examples 4 to 7 are tabulated below:

Qty (mg/ml)

| S. No | Ingredients | Ex 4 | Ex 5 | Ex 6 | Ex 7 |
|---|---|---|---|---|---|
| 1 | Naproxen | 25 | 50 | 2.5 | 5 |
| 2 | SBECD | 30 | 30 | 2.5 | 5 |
| 3 | Sodium Chloride | — | — | 7.8 | 7.5 |
| 4 | Milli Q Water | Qs to | Qs to | Qs to | Qs to |
| | | 1 mL | 1 mL | 1 mL | 1 mL |
| | Colour | 0 | 0 | 0 | 0.001 |
| | Hemolysis | 0 | 0 | 0 | 0 |

The formulation of the present invention comprises naproxen, a cyclodextrin, optionally one or more pH modifying agents and a chelating agent. The formulation has a pH in the range of 6 to 9.

The cyclodextrins may be selected from the group comprising, but not limited to α, β, and γcyclodextrin and cyclodextrins modified with alkyl-, hydroxyalkyl-, dialkyl-, and sulfoalkyl-ether modified cyclodextrins such as methyl or hydroxypropyl β-cyclodextrins (HPβCD), methyl-and-ethyl-β-cyclodextrin, sulfoalkylether-substituted beta-cyclodextrin, sulfobutylether-β-cyclodextrin (SBECD), sugammadex and mixtures.

The pH modifying agents may be selected from the group comprising arginine, L-histidine, glycine, lysine, citric acid, succinic acid, lacto bionic acid, acetic acid, hydrochloric acid and glutamic acid.

The chelating agents may be selected from DOTA (1,4,7,10-tetraazacyclododecane-1,4,7,10-tetraacetic acid), DTPA (diethylene triamine-N,N,N′,N″,N″-penta acetate), EDTA (Ethylene diamine tetra acetic acid) and the like.

In one embodiment, the formulation of the present invention comprises:
(i) naproxen
(ii) a cyclodextrin
(iii) optionally a pH modifying agent, and
(iv) an aqueous solvent

The pharmaceutical formulation of the present invention may comprise pharmaceutically acceptable excipients like buffers, solvents or co solvents, stabilizing agents, solubilizing agents, preservatives, tonicity agents and thereof.

The tonicity agents may be selected from the group comprising dextrose, glycerine, mannitol, potassium chloride and sodium chloride In the present invention these may range from 0 to 5% w/v.

In another embodiment, the formulation comprises:
(i) naproxen
(ii) SBECD
(iii) optionally a pH modifying agent, and
(iv) water for injection

In another embodiment, the formulation comprises:
(i) naproxen
(ii) SBECD
(iii) optionally a pH modifying agent, and
(iv) water for injection, wherein the formulation has >95% of drug remaining throughout its shelf life

The quantity of naproxen in the formulation may range from 0.2 to 10% w/v. The cyclodextrin may be present from 0.2 to 10% by the weight of the formulation. Preferably the formulation may contain naproxen ranging from 0.25 to 5.5% w/v and cyclodextrin may be present from 0.25 to 5.5% w/v.

Yet in another embodiment, the formulation comprises:
(i) naproxen ranges from 0.2 to 10% w/v
(ii) SBECD ranges from 0.2 to 10% w/v
(iii) optionally pH modifying agent selected from the group comprising arginine, L-histidine, glycine, lysine, citric acid, succinic acid, lacto bionic acid, acetic acid, hydrochloric acid, glutamic acid
(iv) optionally chelating agents selected from the group comprising DOTA (1,4,7,10-tetraazacyclododecane-1,4,7,10-tetraacetic acid), DTPA (diethylene triamine-N,N,N′,N″,N″-penta acetate), EDTA (Ethylene diamine tetra acetic acid) or its salts,
   wherein the ratio of naproxen:cyclodextrin ranges from 0.5:1 to 11:1.

A parenteral formulation of naproxen sodium was made with (i) SBECD; (ii) a combination of SBECD and Glycine; (iii) a combination of SBECD and EDTA and (iv) a combination of SBECD, glycine and EDTA in different concentrations. The formulations were checked for their physical description.

55 mg/ml of naproxen sodium was formulated with SBECD ranging from 5 to 100 mg/ml concentration as shown in the table below:

TABLE 4
Formulations with Naproxen sodium and SBECD combination

| | Naproxen | | |
|---|---|---|---|
| | Sodium | SBECD | |
| Batch No. | (mg/mL) | (mg/mL) | Description |
| 002A1 | 55 | 5 | Clear Colourless |
| 002A2 | 55 | 10 | Clear Colourless |
| 002A3 | 55 | 15 | Clear Colourless |
| 002A4 | 55 | 25 | Clear Colourless |
| 002A5 | 55 | 50 | Slight pink colour |
| 002A6 | 55 | 75 | Slight pink colour |
| 002A7 | 55 | 100 | Pale pink colour |

55 mg/ml of naproxen sodium was formulated with SBECD ranging from 5 to 100 mg/ml concentration and 2 mg/ml of glycine as shown in the table below:

TABLE 5
Formulations with Naproxen sodium,
SBECD and glycine combination

| | Naproxen | | | |
|---|---|---|---|---|
| | Sodium | SBECD | Glycine | |
| Batch No. | (mg/mL) | (mg/mL) | (mg/mL) | Description |
| 002B1 | 55 | 5 | 2 | Clear Colourless |
| 002B2 | 55 | 10 | 2 | Clear Colourless |
| 002B3 | 55 | 15 | 2 | Clear Colourless |
| 002B4 | 55 | 25 | 2 | Clear Colourless |
| 002B5 | 55 | 50 | 2 | Slight pink colour |
| 002B6 | 55 | 75 | 2 | Slight pink colour |
| 002B7 | 55 | 100 | 2 | Pale pink colour |

55 mg/ml of naproxen sodium was formulated with SBECD ranging from 5 to 100 mg/ml concentration and 0.1 mg/ml of EDTA as shown in the table below:

TABLE 6
Formulations with Naproxen sodium,
SBECD and EDTA combination

| | Naproxen | | | |
|---|---|---|---|---|
| | Sodium | SBECD | EDTA | |
| Batch No. | (mg/mL) | (mg/mL) | (mg/mL) | Description |
| 002C1 | 55 | 5 | 0.1 | Clear Colourless |
| 002C2 | 55 | 10 | 0.1 | Clear Colourless |
| 002C3 | 55 | 15 | 0.1 | Clear Colourless |
| 002C4 | 55 | 25 | 0.1 | Clear Colourless |
| 002C5 | 55 | 50 | 0.1 | Slight pink colour |
| 002C6 | 55 | 75 | 0.1 | Slight pink colour |
| 002C7 | 55 | 100 | 0.1 | Pale pink colour |

55 mg/ml of naproxen sodium was formulated with SBECD ranging from 5 to 100 mg/ml concentration, 2 mg of glycine and 0.1 mg/ml of EDTA as shown in the table below:

TABLE 7
Formulations with Naproxen sodium,
SBECD, glycine and EDTA combination

| | Naproxen | | | | |
|---|---|---|---|---|---|
| | Sodium | SBECD | Glycine | EDTA | |
| Batch No. | (mg/mL) | (mg/mL) | (mg/mL) | (mg/mL) | Description |
| 002D1 | 55 | 5 | 2 | 0.1 | Clear |
| | | | | | Colourless |
| 002D2 | 55 | 10 | 2 | 0.1 | Clear |
| | | | | | Colourless |
| 002D3 | 55 | 15 | 2 | 0.1 | Clear |
| | | | | | Colourless |
| 002D4 | 55 | 25 | 2 | 0.1 | Clear |
| | | | | | Colourless |
| 002D5 | 55 | 50 | 2 | 0.1 | Slight |
| | | | | | pink colour |
| 002D6 | 55 | 75 | 2 | 0.1 | Slight |
| | | | | | pink colour |
| 002D7 | 55 | 100 | 2 | 0.1 | Pale |
| | | | | | pink colour |

Formulations of naproxen sodium with SBECD and SBECD, glycine combination were found to be physically stable as there was no change in their description upon storage at varied temperatures.

In some aspects, the formulation may contain an additional active ingredient selected from the group comprising but not limited to acetaminophen, duloxetine, ketorolac tromethamine, tramadol, tapentadol, naloxone, naltrexone, pregabalin, tofacitinib, morphine HCl and gabapentin or their combinations.

Following are few examples that do not limit the scope of the present invention and further describe certain specific aspects and embodiments.

### Example 1

| S. No | Ingredients | Qty Per mL (mg) | %(w/v) |
|---|---|---|---|
| 1 | Naproxen Sodium | 55 | 5.5 |
| 2 | SBECD | 5 | 0.5 |
| 3 | Glycine | 2 | 0.2 |
| 4 | Water for injection | Q.S to 1 mL | Q.S to 100% |

Below is the manufacturing procedure for formulating compositions of the present invention:
(1) All the ingredients were dispensed according to the manufacturing formula.
(2) 80% of water for injection (WFI) was taken into a manufacturing vessel. Weighed quantity of cyclodextrin, optionally other ingredients were added and dissolved by stirring on a magnetic stirrer for 5 min at 400 rpm.
(3) Weighed quantity of naproxen sodium and optionally other active ingredients were added to the above solution and stirring was continued for another five minutes at 400 rpm to form a homogenous solution. The final volume was adjusted with water for injection (WFI) followed by 45 min of autoclaving and further analyzed.

The below examples are formulated in the manner similar to example 1.

### Example 2

| S. No | Ingredients | Qty Per mL (mg) | %(w/v) |
|---|---|---|---|
| 1 | Naproxen Sodium | 55 | 5.5 |
| 2 | SBECD | 5 | 0.5 |
| 3 | Water for injection | Q.S to 1 mL | Q.S to 100% |

*Note:
The above formulation was filled in USP Type 1 Clear Glass vials

TABLE 8
Stability data of naproxen sodium formulation
using SBECD at varied temperature conditions.

| | Batch Number | | | | | | |
|---|---|---|---|---|---|---|---|
| | 005A | | | | | | |
| | | Autoclaved | 2-8° C. | 25° C. | 30° C. | 40° C. | 60° C. |
| Condition | Initial | Initial | 12 M | 12 M | 12 M | 6 M | 1 M |
| Description | Clear | Clear | Clear | Clear | Clear | Clear | Light |
| | colorless | colorless | Brown | Brown | Brown | Brown | brown |
| | solution | solution | color | color | color | color | color |
| | | | solution | solution | solution | solution | solutions |
| pH | 8.19 | 8.14 | 8.16 | 8.21 | 8.35 | 8.32 | 8.06 |
| Osmolality | 323 | 335 | 304 | 348 | 348 | 325 | 351 |
| (mOsm/kg) | | | | | | | |
| Assay | 103.8 | 104.4 | 104.5 | 104.8 | 103.9 | 104.8 | 101.7 |
| (% w/w) | | | | | | | |
| Related | | | | | | | |
| Substances | | | | | | | |
| Unknown 1 | ND | ND | 0.03 | 0.03 | 0.03 | 0.03 | ND |
| Unknown 2 | ND | ND | 0.00 | 0.00 | 0.00 | ND | ND |
| Related | ND | ND | 0.00 | 0.00 | 0.00 | 0.01 | ND |
| Compound-A | | | | | | | |
| Unknown 3 | ND | ND | ND | ND | ND | ND | ND |
| Related | ND | ND | 0.00 | 0.00 | 0.01 | ND | ND |
| Compound-L | | | | | | | |
| Total | ND | ND | 0.03 | 0.03 | 0.04 | 0.04 | ND |
| Impurities | | | | | | | |

### Exampple 3

| S. No | Ingredients | Qty Per mL (mg) | %(w/v) |
|---|---|---|---|
| 1 | Naproxen Sodium | 55 | 5.5 |
| 2 | HPBCD | 5 | 0.5 |
| 3 | Glycine | 2 | 0.2 |
| 4 | Water for injection | Q.S to 1 mL | Q.S to 100% |

### Example 4

| S. No | Ingredients | Qty Per mL (mg) | %(w/v) |
|---|---|---|---|
| 1 | Naproxen | 25 | 2.5 |
| 2 | SBECD | 30 | 3.0 |
| 3 | Water for injection | Q.s to 1 mL | Q.s to 100% |

*Note:
The above formulation was filled in USP Type 1 clear glass vials

TABLE 9
Stability data of naproxen sodium
(25 mg/ml) formulation with SBECD

| | Batch No. | | | | |
|---|---|---|---|---|---|
| | #012 | | | | |
| | Condition | | | | |
| | | 25° C. | 30° C. | 40° C. | 60° C. |
| | Initial | 6 M | 6 M | 6 M | 1 M |
| Description | Clear | Clear | Clear | Clear | Clear |
| | Color- | Color- | Color- | Color- | Color- |
| | less | less | less | less | less |
| | Solution | Solution | Solution | Solution | Solution |
| pH | 7.84 | 7.66 | 7.86 | 7.83 | 7.93 |
| Osmolality | 245 | 252 | 259 | 253 | 251 |
| (mOsm/kg) | | | | | |
| Assay | 101.9 | 101 | 100.3 | 100.9 | 98.4 |
| (% w/w) | | | | | |
| Related | | | | | |
| Substances | | | | | |
| Unknown 1 | ND | ND | ND | ND | ND |
| Unknown 2 | ND | 0.01 | 0.01 | 0.01 | ND |
| Related | ND | 0.01 | 0.00 | 0.00 | ND |
| Compound-A | | | | | |
| Unknown 4 | ND | ND | ND | ND | ND |
| Related | ND | 0.00 | 0.00 | 0.00 | ND |
| Compound-L | | | | | |
| Total | ND | 0.02 | 0.01 | 0.01 | ND |
| Impurities | | | | | |

### Example 5

| S. No | Ingredients | Qty Per mL (mg) | %(w/v) |
|---|---|---|---|
| 1 | Naproxen | 50 | 5.0 |
| 2 | SBECD | 30 | 3.0 |
| 3 | Water for injection | Q.s to 1 mL | Q.s to 100% |

*Note:
The above formulation was filled in USP Type 1 clear glass vials

TABLE 10
Stability data of naproxen sodium
(50 mg/ml) formulation with SBECD

| | Batch No. | | | | |
|---|---|---|---|---|---|
| | #016 | | | | |
| | Condition | | | | |
| | | 25° C. | 30° C. | 40° C. | 60° C. |
| | Initial | 6 M | 6 M | 6 M | 1 M |
| Description | Clear | Clear | Clear | Clear | Clear |
| | Color- | Light | Light | Light | Color- |
| | less | Brown | Brown | Brown | less |
| | Solution | Solution | Solution | Solution | Solution |
| pH | 7.95 | 7.89 | 7.90 | 7.87 | 8.03 |
| Osmolality | 397 | 393 | 370 | 395 | 374 |
| (mOsm/kg) | | | | | |
| Assay | 103.3 | 100.1 | 101 | 100.1 | 98.8 |
| (% w/w) | | | | | |
| Related | | | | | |
| Substances | | | | | |
| Unknown 1 | ND | ND | ND | ND | ND |
| Unknown 2 | ND | 0.01 | 0.01 | 0.01 | ND |
| Related | ND | 0.01 | 0.00 | 0.01 | ND |
| Compound-A | | | | | |
| Unknown 4 | ND | ND | ND | ND | ND |
| Related | ND | 0.00 | 0.00 | 0.01 | ND |
| Compound-L | | | | | |
| Total | ND | 0.02 | 0.01 | 0.03 | ND |
| Impurities | | | | | |

### Example 6

| S. No | Ingredients | Qty Per mL (mg) | %(w/v) |
|---|---|---|---|
| 1 | Naproxen | 2.5 | 0.25 |
| 2 | SBECD | 2.5 | 0.25 |
| 3 | Sodium Chloride | 7.8 | 0.78 |
| 4 | Water for injection | Q.s to 1 mL | Q.s to 100% |

*Note:
The above formulation was filled in USP Type 1 clear glass vials

TABLE 11
Stability data of naproxen sodium
(2.5 mg/ml) formulation with SBECD

| | Batch No. | | | | |
|---|---|---|---|---|---|
| | #035 | | | | |
| Condition | Initial | 25° C. 1M | 30° C. 1M | 40° C. 1M | 60° C. 1M |
| Description | Clear | Clear | Clear | Clear | Clear |
| | Color- | Color- | Color- | Color- | Color- |
| | less | less | less | less | less |
| | Solution | Solution | Solution | Solution | Solution |
| pH | 6.93 | 7.1 | 6.96 | 7.00 | 6.89 |
| Osmolality | 276 | 272 | 272 | 281 | 272 |
| (mOsm/kg) | | | | | |
| Assay (% w/w) | 102.1 | 101.3 | 100.7 | 102.0 | 101.7 |
| Related | | | | | |
| Substances | | | | | |
| Unknown 1 | 0.00 | 0.00 | 0.00 | 0.01 | 0.00 |
| Unknown 2 | 0.01 | 0.01 | 0.01 | ND | 0.01 |
| Related | 0.00 | 0.00 | 0.01 | 0.01 | 0.01 |
| Compound-A | | | | | |
| Related | 0.01 | 0.00 | 0.01 | 0.01 | 0.01 |
| Compound-L | | | | | |
| Total | 0.02 | 0.01 | 0.03 | 0.03 | 0.03 |
| Impurities | | | | | |

### Example 7

| S. No | Ingredients | Qty Per mL (mg) | %(w/v) |
|---|---|---|---|
| 1 | Naproxen | 5 | 0.5 |
| 2 | SBECD | 5 | 0.5 |
| 3 | Sodium Chloride | 7.5 | 0.75 |
| 4 | Water for injection | Q.s to 1 mL | Q.s to 100% |

*Note:
The above formulation was filled in USP Type 1 clear glass vials

TABLE 12
Stability data of naproxen sodium
(5 mg/ml) formulation with SBECD

| | Batch No. | | | | |
|---|---|---|---|---|---|
| | #032 | | | | |
| Condition | Initial | 25° C. 1M | 30° C. 1M | 40° C. 1M | 60° C. 1M |
| Description | Clear | Clear | Clear | Clear | Clear |
| | Color- | Color- | Color- | Color- | Color- |
| | less | less | less | less | less |
| | Solution | Solution | Solution | Solution | Solution |
| pH | 7.07 | 7.26 | 7.09 | 7.29 | 7.05 |
| Osmolality | 297 | 294 | 291 | 294 | 285 |
| (mOsm/kg) | | | | | |
| Assay (% w/w) | 101.5 | 101.3 | 102.8 | 101.6 | 101.1 |
| Related | | | | | |
| Substances | | | | | |
| Unknown 1 | 0.00 | 0.00 | 0.00 | 0.00 | 0.01 |
| Unknown 2 | 0.01 | 0.01 | 0.01 | 0.01 | ND |
| Related | 0.01 | 0.00 | 0.00 | 0.01 | 0.01 |
| Compound-A | | | | | |
| Related | 0.01 | 0.01 | 0.00 | 0.01 | 0.01 |
| Compound-L | | | | | |
| Total | 0.03 | 0.02 | 0.01 | 0.03 | 0.03 |
| Impurities | | | | | |

### Example 8

| S. No | Ingredients | Qty Per mL (mg) | %(w/v) |
|---|---|---|---|
| 1 | Naproxen | 25 | 2.5 |
| 2 | Acetaminophen | 10 | 1.0 |
| 3 | SBECD | 30 | 3.0 |
| 4 | EDTA | 0.2 | 0.02 |
| 5 | Water for injection | Qs to 1 mL | Q.s to 100% |

### Example 9

| S. No | Ingredients | Qty Per mL (mg) | %(w/v) |
|---|---|---|---|
| 1 | Naproxen | 25 | 2.5 |
| 2 | Duloxetine HCl | 5 | 0.5 |
| 3 | SBECD | 30 | 3.0 |
| 4 | Water for injection | Qs to 1 mL | Q.s to 100% |

### Example 10

| S. No | Ingredients | Qty Per mL (mg) | %(w/v) |
|---|---|---|---|
| 1 | Naproxen | 25 | 2.5 |
| 2 | Ketorolac | 15 | 1.5 |
| | tromethamine | | |
| 3 | SBECD | 30 | 3.0 |
| 4 | Water for injection | Qs to 1 mL | Q.s to 100% |

### Example 11

| S. No | Ingredients | Qty Per mL (mg) | %(w/v) |
|---|---|---|---|
| 1 | Naproxen | 25 | 2.5 |
| 2 | Tramadol | 25 | 2.5 |
| 3 | SBECD | 30 | 3.0 |
| 4 | Water for injection | Qs to 1 mL | Q.s to 100% |

### Example 12

| S. No | Ingredients | Qty Per mL (mg) | %(w/v) |
|---|---|---|---|
| 1 | Naproxen | 25 | 2.5 |
| 2 | Tapentadol | 25 | 2.5 |
| 3 | SBECD | 30 | 3.0 |
| 4 | Water for injection | Qs to 1 mL | Q.s to 100% |

### Example 13

| S. No | Ingredients | Qty Per mL (mg) | %(w/v) |
|---|---|---|---|
| 1 | Naproxen | 25 | 2.5 |
| 2 | Naloxone | 5 | 0.5 |
| 3 | SBECD | 30 | 3.0 |
| 4 | Water for injection | Qs to 1 mL | Q.s to 100% |

### Example 14

| S. No | Ingredients | Qty Per mL (mg) | %(w/v) |
|---|---|---|---|
| 1 | Naproxen | 25 | 2.5 |
| 2 | Naltrexone | 50 | 5.0 |
| 3 | SBECD | 30 | 3.0 |
| 4 | Water for injection | Qs to 1 mL | Q.s to 100% |

### Example 15

| S. No | Ingredients | Qty Per mL (mg) | %(w/V) |
|---|---|---|---|
| 1 | Naproxen | 25 | 2.5 |
| 2 | Pregabalin | 25 | 2.5 |
| 3 | SBECD | 30 | 3.0 |
| 4 | Water for injection | Qs to 1 mL | Q.s to 100% |

### Example 16

| S. No | Ingredients | Qty Per mL (mg) | %(w/v) |
|---|---|---|---|
| 1 | Naproxen | 25 | 2.5 |
| 2 | Tofacitinib | 20 | 2.0 |
| 3 | SBECD | 30 | 3.0 |
| 4 | Water for injection | Qs to 1 mL | Q.s to 100% |

### Example 17

| S. No | Ingredients | Qty Per mL (mg) | %(w/v) |
|---|---|---|---|
| 1 | Naproxen | 25 | 2.5 |
| 2 | Morphine | 25 | 2.5 |
| 3 | SBECD | 30 | 3.0 |
| 4 | Water for injection | Qs to 1 mL | Q.s to 100% |

### Example 18

| S. No | Ingredients | Qty Per mL (mg) | %(w/v) |
|---|---|---|---|
| 1 | Naproxen | 25 | 2.5 |
| 2 | Gabapentin | 25 | 2.5 |
| 3 | SBECD | 30 | 3.0 |
| 4 | Water for injection | Qs to 1 mL | Q.s to 100% |

1. A stable liquid parenteral formulation of naproxen comprising of
(i) naproxen,
(ii) cyclodextrin,
(iii) optionally a pH modifying agent, and
(iv) an aqueous solvent

2. The formulation of claim 1, wherein pH modifying agent is selected from the group comprising arginine, L-histidine, glycine, lysine, citric acid, succinic acid, lacto bionic acid, acetic acid, hydrochloric acid and glutamic acid.

3. The formulation of claim 1, wherein naproxen is present in the range of 0.2% w/v to 10% w/v based on the total weight of the formulation.

4. The formulation of claim 1, wherein cyclodextrin is present in the range of 0.2% w/v to 10% w/v based on the total weight of the formulation.

5. The formulation of claim 1 further comprising a chelating agent selected from the selected from the group comprising: DOTA (1,4,7,10-tetraazacyclododecane-1,4,7,10-tetraacetic acid), DTPA (diethylene triamine-N,N,N′,N″,N″-penta acetate), EDTA (Ethylene diamine tetra acetic acid) and the like.

6. The formulation of claim 1 further relates to method of treating pain using composition as claimed in claim 1.

7. A stable liquid parenteral formulation of naproxen comprising of:
(i) naproxen,
(ii) SBECD,
(iii) a pH modifying agent
(iv) optionally a chelating agent, and
(v) water for injection
wherein the ratio of naproxen:cyclodextrin ranges from 0.5:1 to 11:1.

8. The formulation of claim 7, wherein naproxen is present in the range of 0.2% w/v to 10% w/v based on the total weight of the formulation.

9. The formulation of claim 7, wherein SBECD is present in the range of 0.2% w/v to 10% w/v based on the total weight of the formulation.

10. The formulation of claim 7, wherein the drug content is more than 95% throughout its shelf life.

11. The formulation of claim 7, wherein the total impurities are less than 0.5% when placed at recommended storage conditions.

12. The liquid parenteral formulation of naproxen of claim 7, wherein the formulation is non-haemolytic.

13. A stable liquid parenteral formulation of naproxen comprising
(i) naproxen in a concentration of 0.2% w/v to 10% w/v based on total weight of the formulation,
(ii) SBECD in a concentration of 0.2% w/v to 10% w/v based on total weight of the formulation,
(iii) a pH modifying agent selected from the group comprising arginine, L-histidine, glycine, lysine, citric acid, succinic acid, lacto bionic acid, acetic acid, hydrochloric acid and glutamic acid,
(iv) optionally a chelating agent, and
(v) water for injection
wherein the ratio of naproxen:cyclodextrin ranges from 0.5:1 to 11:1.

14. A method of treating pain using a liquid parenteral formulation of naproxen comprising naproxen, a cyclodextrin and optionally a pH modifying agent.

15. A stable liquid parenteral formulations of naproxen according to claim 1, wherein formulation further comprises of additional active ingredients.
